(19) United States
(12) Patent Application Publication
(10) Pub. No.: US 2024/0412360 A1
(43) Pub. Date: Dec. 12, 2024

(54) ULTRASOUND-GUIDED SPINAL INJECTIONS

(71) Applicants: Frank J. Kosarek, Charlotte, NC (US); Michelle L. Rivera, Charlotte, NC (US)

(72) Inventors: Frank J. Kosarek, Charlotte, NC (US); Michelle L. Rivera, Charlotte, NC (US)

(21) Appl. No.: 18/736,452

(22) Filed: Jun. 6, 2024

Related U.S. Application Data
(60) Provisional application No. 63/471,800, filed on Jun. 8, 2023.

(51) Int. Cl. G06T 7/00; A61B 17/34; A61B 34/10; G16H 10/60

(52) U.S. Cl. CPC G06T 7/0012; A61B 17/3403; G16H 10/60; A61B 2034/107; G06T 2207/10132; G06T 2207/20084

(57) ABSTRACT

Two related systems and methods of identifying acoustic windows for spinal injections are provided. Each comprises receiving a series of two-dimensional ultrasound images and feeding them into a trained neural network to identify bone and other features. In one, a three-dimensional image of vertebrae in the spine is generated and an acoustic window is identified between the posterior osseous structures of two vertebral body segments. In the other, a spinous process of a patient's vertebra is identified. Using medical and demographic data concerning the patient, a location of an acoustic window with respect to the spinous process is estimated. In each method, a human user is then guided to penetrate with an injection needle at the acoustic window and to inject within a space in the spine while avoiding encountering bone or neurovascular structure.

### CROSS-REFERENCE TO RELATED APPLICATIONS

This application is a non-provisional application claiming priority to provisional U.S. App. No. 63/471,800, also titled “ULTRASOUND-GUIDED SPINAL INJECTIONS” and filed on Jun. 8, 2023, which is hereby incorporated by reference in its entirety.

### FIELD OF INVENTION

This disclosure relates to methods of automatically processing image data to guide during medical procedures, and more specifically, to systems and methods for processing ultrasound imaging to automatically identify acoustic windows between the posterior osseous structures of the spine as injection sites. These acoustic windows will serve as entry portals for injections into the epidural space and the spinal canal.

### BACKGROUND

There are a variety of medical procedures affecting the spine or spinal cord, such as epidural anesthetic, epidural neurostimulator insertion or steroidal injections, that require identification of the best path of entry to avoid the injections needle engagement with the posterior osseous structures of the spine. A needle encountering the spine may cause injury or unnecessary pain to the patient and/or require withdrawal of the needle and a second puncture. Avoidance of important neurovascular structures along the path of injection is also an issue when performing these procedures.

Historically, to identify a window between the posterior osseous structures of the spine for the injection, the operator would use their experience and anatomic landmarks to try to gain access to the spinal canal. Alternatively, 2D or 3D image guided fluoroscopy can be utilized to help the operator to the appropriate location for needle insertion into the spinal canal. However, these methods can expose patients, the operator and the fluoroscopy team to significant levels of radiation, are subject to the experience and capabilities of the operator and can require expensive, bulky equipment. In contrast to fluoroscopically guided spine injection, ultrasound guided injections do not involve ionizing radiation and are more readily available than other imaging systems previously used. However, ultrasound has its own limitations, including, but not limited to: 1) ultrasound is very user dependent and requires the user to have significant training and experience with ultrasound injections and ultrasound guided needle guidance and 2) ultrasound creates two-dimensional images with acoustic shadows that are more difficult to convert to the precise three-dimensional image data.

Existing ultrasound-guided spinal injections generally require significant expertise by the individual operating the injection needle. The operator must identify a window for entry (an “acoustic window”) between the posterior osseous structures based on personal interpretation of the ultrasound as well as translate that knowledge into a proper location and orientation for the needle to enter the back. Procedures may be unnecessarily delayed by waiting for a person with this expertise to be available. In some cases, an expert may not be available at all, resulting in non-performance of the procedure, with potential detrimental consequences to the patient.

As a result, there are advantages to developing methods to identify acoustic windows between the posterior osseous structures that drastically reduce the risk of pain, complications, and injury associated with spinal injections into the epidural space or spinal canal. There are also advantages to developing injection-aiding systems that would permit a human user with less surgical experience (such as a physician's assistant, nurse, paramedic, or individual with no medical training at all) to be able to perform injections accurately in an emergency or when a doctor or other expert is unavailable or cannot conveniently be present.

### SUMMARY OF THE INVENTION

In order to address the limitations of previous systems in this field, a first new method for identifying acoustic windows for spinal injections is provided. The method comprises receiving a series of two-dimensional ultrasound images associated with a known position and orientation of the ultrasound probe at the moment that each two-dimensional ultrasound image is generated, feeding the two-dimensional ultrasound images into a trained neural network to identify bone and other features in the two-dimensional ultrasound images, using the identified features and positions and orientations of the ultrasound probe at the moment the images were generated to generate a three-dimensional image of vertebrae in the spine, using a neural network to identify an acoustic window between the posterior osseous structures of two vertebral body segments, and guiding a human user to penetrate with a navigated injection needle—having an electromagnetic sensor (EM) or optical tracker (OT) attached that can be seen on a user interface (UI)—into the acoustic window to inject within a space in the spine without the injection needle encountering bone or neurovascular structure.

A second new method of identifying acoustic windows in the spine of a patient is also provided. The method comprises feeding the two-dimensional ultrasound images into a trained neural network to identify bone and other features in the two-dimensional ultrasound images; using the identified features, identifying a spinous process of a patient's vertebra; receiving medical and demographic data concerning the patient; automatically determining a likely location of an acoustic window with respect to the spinous process; and guiding a human user to penetrate with an injection needle at the acoustic window and to inject within a space in the spine without the injection needle encountering bone.

A navigated needle can be introduced into the navigated 3D ultrasound field and this navigated needle can be seen in the field of the 3D point cloud of the bony surface of the spine. The needle can be visualized on the UI in real time with tip entering the thecal sac.

Moreover, this same process of spinal injection can be used for any needle injection into the spine which would include facet injections, vertebroplasty, nerve root blocks, grey ramus blocks, and any and all types of spinal injections, not just limited to lumbar punctures and epidural injections.

Particular embodiments of the disclosed methods may include variants where the human user has sight augmented by glasses or a helmet that visually indicate the acoustic window, or where the injection device uses visual, auditory, or haptic feedback to indicate an injection site for the acoustic window.

### BRIEF DESCRIPTION OF THE DRAWINGS

Other aspects, features and advantages will become more fully apparent from the following detailed description, the appended claims, and the accompanying drawings (provided solely for purposes of illustration without restricting the scope of any embodiment), of which:

FIGS. 1A, 1B, and IC depict, in simplified form, a possible setup of devices in a clinic to gather ultrasound data and prepare for an injection;

FIG. 2 depicts, in simplified form, a flowchart of a method for generating ultrasound data, building a model that identifies the acoustic windows in a particular patient's spine, and providing feedback to a person performing an injection;

FIG. 3 depicts an exemplary three-dimensional coronal reconstruction that could be generated during the method illustrated in FIG. 2;

FIG. 4 depicts, in simplified form, a flowchart for an alternative method for locating an injection site without access to metadata for each ultrasound's location and orientation, based on individual two-dimensional scans being considered independently, rather than building a three-dimensional model;

FIG. 5A depicts a fictional ultrasound image;

FIG. 5B depicts the same ultrasound image having been annotated for training of a machine learning system;

FIG. 6A depicts an example feature identification by a machine learning system;

FIG. 6B depicts an error determination step during training of the machine learning system;

FIG. 7 depicts, in simplified form, a method for evaluation of the machine learning output and back-propagating the error to reduce future error in classification;

FIG. 8 depicts an exemplary neural network machine learning structure to train in structure identification and classification; and

FIG. 9 depicts another example use of the system in a clinical environment.

FIG. 10 depicts a navigated needle with EM tracker attached as the preferred way of live navigation of the needle with a user interface.

FIG. 11 depicts the navigated needle displayed on the user interface in real time as it is seen traversing the soft tissues to enter the thecal sac through the acoustic window (seen on three-dimensional point cloud of bony surface) of a spine.

### DETAILED DESCRIPTION OF THE DRAWINGS

To address the limitations in prior art systems, a system is disclosed to automatically interpret ultrasound data, to identify acoustic windows between vertebrae via real-time analysis by a trained machine learning system, and to provide precise guidance to a human user in locating an injection path into the spine.

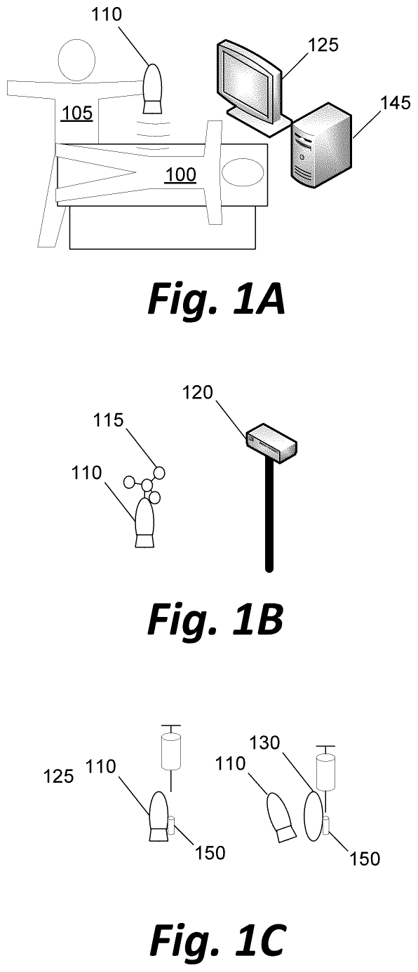

FIGS. 1A, 1B, and IC depict, in simplified form, a possible setup of devices in a clinic to gather ultrasound data and prepare for an injection.

As depicted in FIG. 1A, in order to begin gathering data to target an injection, a patient 100 is scanned by an operator 105 with a handheld ultrasound probe 110.

In one embodiment, shown in FIG. 1B, the handheld ultrasound probe 110 includes a set of pinpoint indicators 115 that can be tracked by a receiver 120. In one embodiment, the receiver 115 picks up infrared light emitted by light emitting diodes (LEDs) at each pinpoint indicator 120; in other embodiments, the receiver 120 may rely on visible light or another spectrum or means of sensing, and the pinpoint indicators may generate or reflect signals in that medium to be distinguished from the background. Because there are three or more such indicators 115 in a pre-arranged configuration, the receiver 120 can calculate, from the apparent distances and angles between them, the location and orientation of the ultrasound probe 110 in space. Alternatively, in another embodiment often preferred, an electromagnetic tracking system may be used such that a sensor on the probe itself is constantly measuring its current orientation—or a navigated needle attached to the probe is located—by reference to one or more generators of a signal in the room and other fiducial markers like the vector of gravitational pull, of magnetic north, or other stable reference points. Ultimately, it does not matter whether a calibrated external sensor tracks the ultrasound probe 110, or if instead an internal accelerometer or electromagnetic sensor with source or other sensor within the ultrasound probe 110 is used, so long as there is a means for determining the probe's location and orientation by sensing the probe's movement and rotations along three axes.

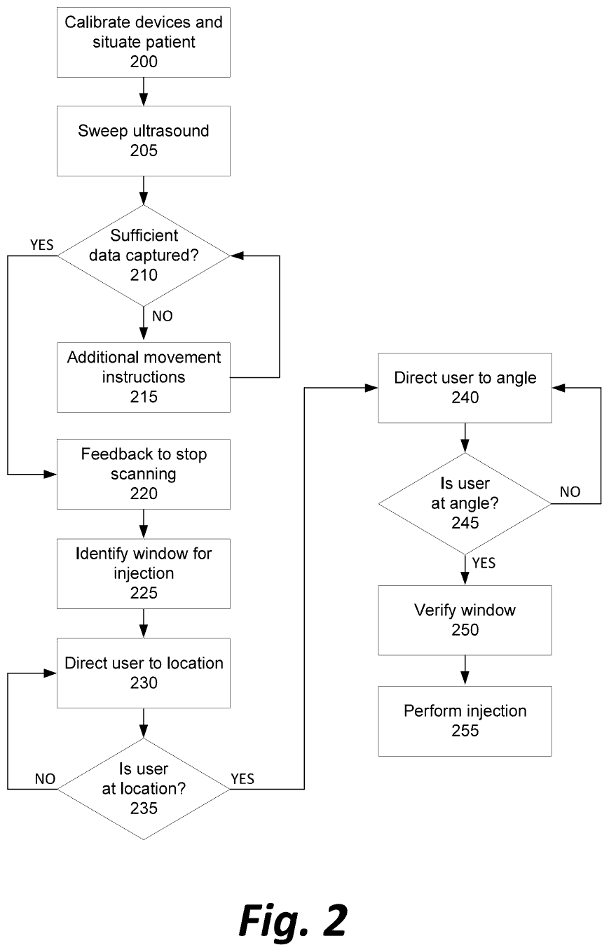

Using this setup, a large number of overlapping two-dimensional ultrasounds, each accompanied by metadata recording the location and orientation at which the image data was received, can be consolidated to build a three-dimensional model of the patient's spine. This method does require proper placement of the receiver 120 with respect to the patient 100 and may require a lengthy calibration process to verify that the probe's location and orientation are being accurately tracked, before useful ultrasound data can be gathered. The receiver 120 may be selected from among commercially available devices such as the preferred method using an EM tracker such as the Northern Digital Inc. Aurora® EM tracking solution or Polaris Lyra® or a similar optical navigation device. The process of obtaining data to correlate the separate two-dimensional scans into a comprehensive three-dimensional model is discussed in further detail below, in association with FIG. 2.

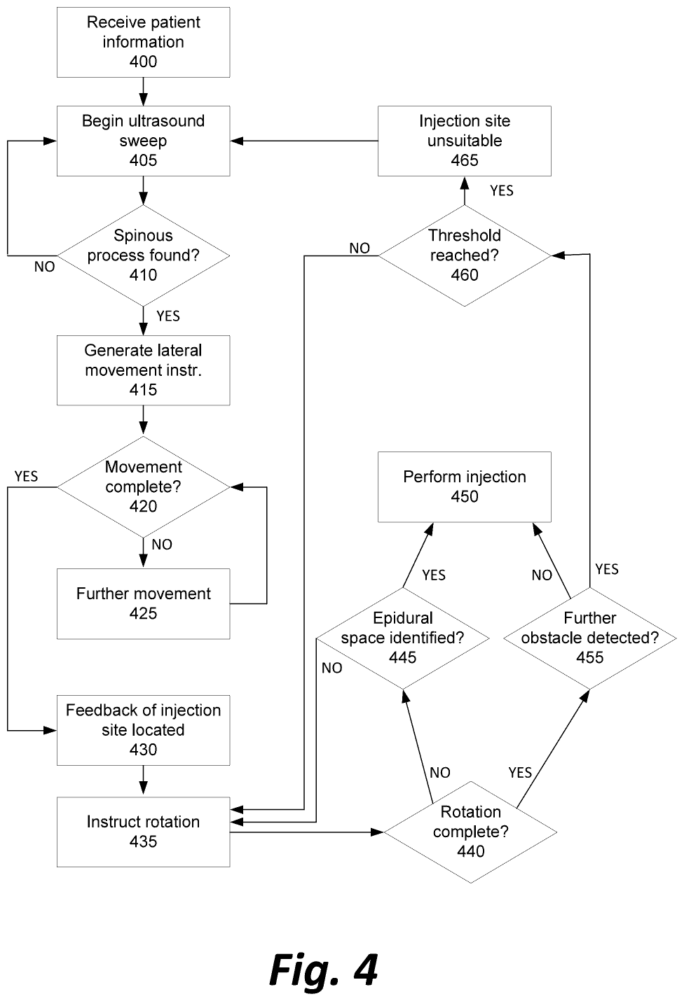

In a second embodiment, no form of external or internal location/orientation tracking may be used, and the probe 110 will generate two-dimensional image data without metadata for each image's location and orientation, according to a method described in further detail below, in association with FIG. 4. As a result, there may be no means to connect the separate individual images into a more comprehensive three-dimensional model, but each image may still be used as input to a trained machine learning system that is expecting a spinal ultrasound image and will try to determine useful information from it. The unique ultrasonographic shape, location, depth from the skin and spatial relation of the spinal elements relative to each other, can enable a neural network to correctly identify the spinous process, the lamina, the facets and transverse processes of the posterior elements of the spine. The distances between these posterior elements of the vertebral bodies can be predicted based upon a large cohort of patients' spines introduced into a neural network. Slight differences in the distances between the spinal elements can be expected based on age, sex, level of the spine and BMI, and this data can be entered into the neural network's calculation of the spatial relationship of the posterior spinal structures. A scan is first performed in the sagittal plane parallel along the spinous processes. A neural network will identify their presence, and based on the demographics of the patient, and known distances between the posterior osseous elements of the spine, instruct the operator to move the ultrasound probe a certain distance from midline. The distance to move the probe from the midline will be displayed to the operator, and the operator can move the probe laterally the suggested distance using a laser guided distance, gyroscope or other method which will record the distance that the operator has moved the probe from midline. In this new location, the neural network will then identify the lamina and facets of the vertebral body. If the spinous processes are still seen and the lamina not seen, the system will instruct the operator to move the probe laterally from midline. If the lamina is not seen, but the transverse process is seen, the system will instruct the operator to move the probe more towards the midline. Once the lamina and facets are identified, the operator will tilt the probe approximately 30 degrees towards the midline until the acoustic window is identified using the AI algorithm. The algorithm will also search for the presence of the anterior and posterior epidural hyperechoic acoustic reflection, pathognomonic of identification of the epidural space anterior and posterior to the thecal sac. The AI algorithm will give feedback to the operator once the acoustic window is seen. The acoustic window will allow ultrasound waves to enter the spinal canal and will allow identification of the anterior and the posterior epidural space.

These structures will be identified by the Al algorithm as verification of acoustic window identification and proper location for needle entrance into the spinal canal. A needle, via use of a needle guide, can then be inserted into the spine through the acoustic window.

In both of these embodiments, a preferred format for the ultrasound image is brightness mode (“B mode”) grayscale imaging that has been modulated and compressed from the raw, “pre-beamformed” data captured by the ultrasound probe 110. This format will typically be the best for annotation of data by a human expert as input to a machine learning system. However, in other embodiments, it could be that all analysis would instead rely on the raw, pre-beamformed data: the radio frequency (RF) or in-phase quadrature (IQ) data. The RF/IQ data will have a wider spectrum than the grayscale brightness imaging, and encode more actual data that a machine learning system can take advantage of. As a result, if a machine learning system can be trained on raw or RF/IQ data, it may have more sophisticated bone surface detection capabilities than a system trained on annotated B mode data alone.

Regardless of embodiment, the ultimate goal is not only to transmit ultrasound data in real time to a display 125 for evaluation by the operator, but also to transmit the ultrasound data to a computer processor for an automatic, real-time evaluation that identifies spinous processes, transverse processes, facets, and laminae, and other features of the vertebrae, and that identifies an acoustic window, a gap between the posterior bony elements of two vertebral bodies that is especially suitable for a spinal injection. The identification of the hyperechoic reflection of the anterior and posterior epidural space is also needed to verify the acoustic window into the spinal canal. The processing of the data to achieve this goal and the means by which the operator is guided are discussed further below. In a preferred embodiment, the probe 110 is Bluetooth-enabled to transmit ultrasound data wirelessly rather than relying on a data cable.

In some embodiments, as depicted in FIG. 1C, the probe 110 may itself be fitted with a navigated injection needle guide 150, so that—once properly placed—the navigated needle will be seen on a user interface entering the patient's body along a path that the probe and navigated needle receiver can verify in real time to be aligned with an acoustic window. Live feedback on the UI can be seen because the needle is navigated with attached EM or Optical tracker and can be seen in real time live on the UI entering the patients spinal canal and thecal sac. In other embodiments, a separate injection needle device 130 may be used in conjunction with the probe 110. In either case, the device with the injection needle will include a form of sensory feedback to indicate that the device is properly aligned and ready for injection. This feedback may be visual, such as the color of a light, a blinking of the light, whether the light is on or off, or other patterns of light production; it may be auditory, such as a tone, beep, sound effect, or verbal instruction; or it may be haptic, such as a vibration, rumble, or pattern of movements. These forms of visual, audio, or haptic feedback will also be used in many future steps, as mentioned below. Feedback is also provided, through the navigated injection needle device and/or the display 125, to indicate movement necessary to find the injection site, such as an instruction to move left/right or up/down, based on the current location of the injection needle with respect to a previously identified acoustic window. A robotic arm could also guide the placement of the injection needle.

Feedback may alternatively be provided by a separate wearable device 135, such as glasses or goggles with an augmented reality (AR) interface that visually indicates instructions or an injection site, headphones or other speakers that produce audible feedback, or a worn bracelet or other device that can provide haptic feedback with vibrations.

Graphics in the display 125 and the feedback in the probe 110, other injection needle device 130, or other wearable device 140 are preferably generated by a server computing device 145 that is receiving the ultrasound data from the probe 110 and the location/orientation data from the probe 110 or receiver 120 and feeding this information into a trained neural network in order to identify features and to augment displays or provide the desired feedback, according to processes described in greater detail below. Although this disclosure proceeds under the assumption that the server computing device 145 to which the ultrasound data is transmitted is a physically separate device, it is conceivable that a processor and memory would be incorporated into the ultrasound probe 110 or the separate injection needle device 130. In such an embodiment, rather than wireless data transmission to or from the server, the server could receive or send data via a wired connection to the other components of the device, or by writing to or reading from a shared memory.

FIG. 2 depicts a flowchart of generating ultrasound data, building a model that identifies one or more acoustic windows in a particular patient's spine, and provides feedback to a person performing an injection.

As an initial step, any calibration of ultrasound devices and placing the patient into a stable, predefined location are completed (Step 200). This includes, for example, setting up the receiver 120 and ensuring that it accurately reads the location of the ultrasound probe 110 with respect to the patient's body, and ensuring that the patient is in a comfortable position and will not need to shift during the scan, ruining the ability to patch together scans assumed to be at the same coordinates with respect to the patient's body.

Next, the ultrasound probe 110 is swept across the patient's lower back (Step 205), starting in the midline over the spinous process region. Ultrasound data begins to be transmitted from the probe 110 to the server 145.

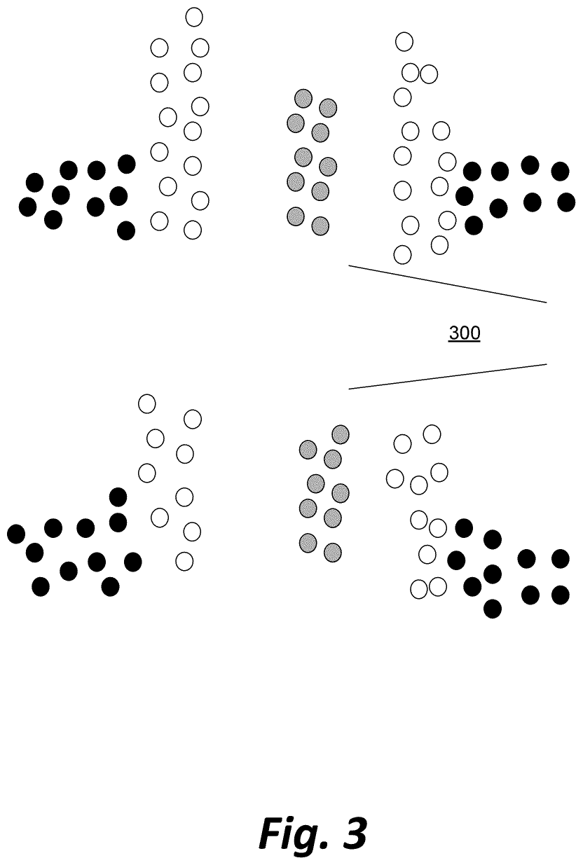

After a single sweep, not enough data is likely to have been gathered to generate a comprehensive three-dimensional point cloud of the patient's spine (see FIG. 3). When, at a future time, the cloud has been sufficiently captured (Step 210), the initial gathering process is completed. If the cloud is not sufficiently captured, the operator is directed to move the ultrasound probe left, right, superiorly and inferiorly over the spine (Step 215), and the comprehensiveness of the point cloud is re-evaluated (back to Step 210).

At the moment that the point cloud is complete, the operator receives feedback (visual, auditory, or haptic) that enough scanning has been performed (Step 220).

FIG. 3 depicts an exemplary three-dimensional coronal reconstruction that could be generated during the method illustrated in FIG. 2.

Each point gathered may be color coded according to tissue type or function, as predicted by the machine learning algorithm. For example, though FIG. 3 is only rendered in grayscale, different colors may be used for laminae, spinous processes, and so on. As depicted in FIG. 3, transverse processes are rendered in black, spinous processes are rendered in gray, and laminae or facets are rendered in white. As the model is filled in by successive passes with the ultrasound probe, an acoustic window 300 (depicted in FIG. 3 as bounded by solid lines, between two vertebrae) will become apparent from the lack of data points automatically identified as bone in that region, compared to other regions over which the ultrasound probe passes.

Returning to FIG. 2, after the model is complete, an optimal location and angle for the injection will be automatically determined (Step 225) by the machine learning system or by another system configured to receive the model as input, identifying an injection path comprising a location and angle that avoids passing too closely to any of the points identified as bone in the point cloud. In some embodiments, when the machine learning classifier has been trained to perform identification of posterior or anterior epidural spaces, the location and angle may be specifically determined to match the location and angle that the ultrasound probe had been at when such epidural spaces were visible—this implies that the ultrasound probe had been aligned with an acoustic window that revealed such epidural spaces. The identification of an epidural space creates a virtual certainty that a path will not traverse any bone, as bone will overshadow and obscure a posterior or anterior epidural space.

The user will then be guided to the injection site (Step 230), preferably by a combination of initial audiovisual input and corrective guidance after movement of the injection guide begins. For example, a display might read “Please move the probe 2.5 cm to your right, and 1 cm down.” As the user moves the probe, a loop will continue of checking the correctness of the location (Step 235) and providing refined guidance (e.g., “You have passed the point. Please move the probe 1 cm to the left.”).

After the correct location has been reached, the user will be similarly guided to the correct injection angle (Step 240), again preferably by a combination of initial audiovisual input and a loop of corrective guidance after movement of the injection guide begins (Steps 245 and back to 240). When the angle appears to have been reached, there will be an attempted verification of the correctness of all prior analysis (Step 250). This verification would preferably include identifying that a posterior and/or anterior epidural space is visible on the ultrasound.

Finally, the injection is performed (Step 255). In one embodiment, this is done using the needle guide 150 depicted in FIG. 1C, while the ultrasound probe is con

FIG. 4 depicts an alternative method for locating an injection site without access to metadata for each ultrasound's location and orientation, based only on individual two-dimensional scans being considered independently, rather than building a three-dimensional model.

First, information concerning the patient is input by a human operator (Step 400). This information might include, among other data points, the age, sex, body mass index (BMI), portion of the spine imaged, whether the portion of the spine is cervical, thoracic or lumbar, or any other quality of the human patient or the intended scan parameters that could be used to predict the size, depth, and alignment of the spine under the surface of the skin.

Next, the operator begins sweeping the lower back with an ultrasound probe (Step 405) with the intent of aligning the probe parallel along the spinous process of a vertebra adjacent to the intended injection site. At each moment during the sweep, the current scan image from the ultrasound probe is sent to the machine learning classifier to determine whether the image appears to contain the spinous process (Step 410). If the scan does not appear to contain the spinous process, the sweep continues (back to Step 405).

When the scan does detect the spinous process, the human operator is instructed to stop the sweep and is given precise estimated instructions to an injection location (Step 415). For example, a text pop-up on a display visible to the human operator might read “Please move 2.5 cm to the right,” or a machine generated voice might provide the same information audibly. The exact value included in this move instruction is preferably an estimate determined based on the information about the human patient that was previously inserted, based on the statistically most likely distance from the spinous process to an acoustic window in similarly situated patients. Preferably, a machine learning system will be trained to perform estimates when an exact match for a combination of characteristics has never before been cataloged.

As the human operator moves the ultrasound probe towards the intended injection point, the progress of the probe towards that point is tracked (Steps 420 and 425, in a loop). This tracking is preferably performed based on an accelerometer affixed to the ultrasound probe, but might alternatively be performed based on an external camera tracking the probe's location, or by another rangefinding or locating technology.

When the proper location for the injection has been reached, feedback is provided to the human operator (Step 430). This feedback may be visual, auditory, or haptic.

The human operator is further instructed to rotate the ultrasound probe to face inward toward the spine from the off-center injection site (Step 435). This instruction might similarly take the form of a text pop-up reading “Please turn 30 degrees to direct the probe back toward the spine,” or a machine generated voice might provide the same information audibly. The exact angle may, like the distance from the spinous process, be decided based on the obesity or other qualities of the patient.

As the human operator rotates the ultrasound probe towards the intended injection angle, even before the intended angle is reached (Step 440), the ultrasound probe also continues to send data to the machine learning system for analysis. If the machine learning analysis identifies a clearly identified epidural space (Step 445), this indicates a suitable location for injection, even if it is a different angle from the originally computed/estimated angle.

When the intended angle is reached, if the ultrasound probe in conjunction with the machine learning senses a bone surface in the path of the injection (Step 455), the human operator may be alerted and the injection will not be instructed. If this has not occurred more than a predetermined number of times (Step 460), a new injection angle will be tried (back to Step 435). If multiple attempts at locating a window have failed, the injection site is deemed unsuitable (Step 465), and the user will be directed to return to the spinal process (back to Step 405) with the intent of identifying a new injection site relative to the spinous process, different from the previously estimated injection site.

When the proper angle is reached, the injection will be performed along the identified path (Step 450). The injection needle could be guided using a guide, attached or not attached to the ultrasound probe. Alternatively, the needle could also be guided by the operator placing a mark on the location of the skin, setting aside the ultrasound probe, and returning to the patient a moment later. The angle to be used could also be saved in the memory of the ultrasound probe and reproduced at the time when the operator is ready to perform the injection. As a reference the ultrasound probe could use a fiducial such as magnetic north or gravitational pull.

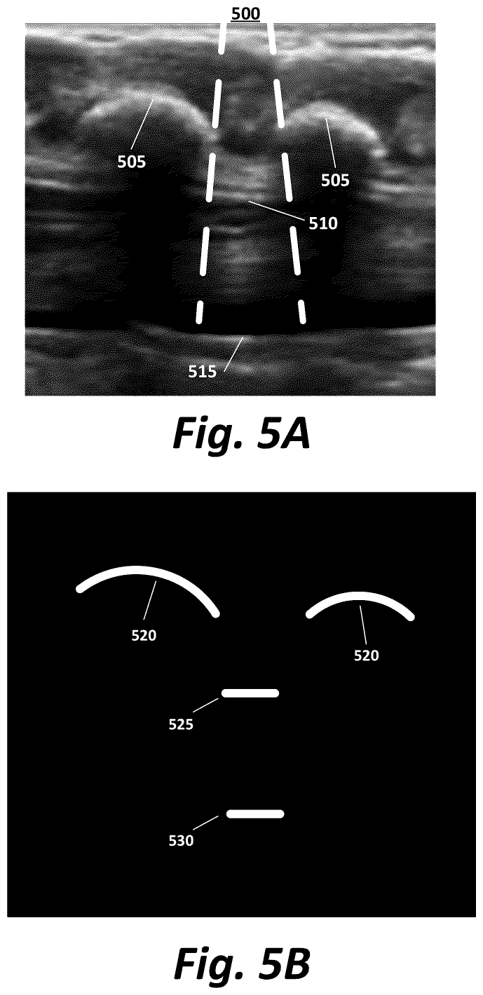

FIG. 5A depicts a fictional ultrasound image.

A human viewer will be able to determine with case that an acoustic window 500 exists between the facets or lamina 505 of two vertebrae (bounded, in the illustration, by white dashed lines). A number of additional features may be picked up by the ultrasound, such as a posterior epidural space 510, an anterior epidural space 515, spinous processes, transverse processes, or other anatomical features. In order to train a machine learning process to have a similar intuition to a human when viewing the same image data, a human expert will use a stylus on a touchscreen to identify and trace these relevant features, with the results of this tracing being shown in FIG. 5B.

FIG. 5B depicts an additional layer to be added to the ultrasound image of FIG. 5A, with annotations for training of a machine learning system.

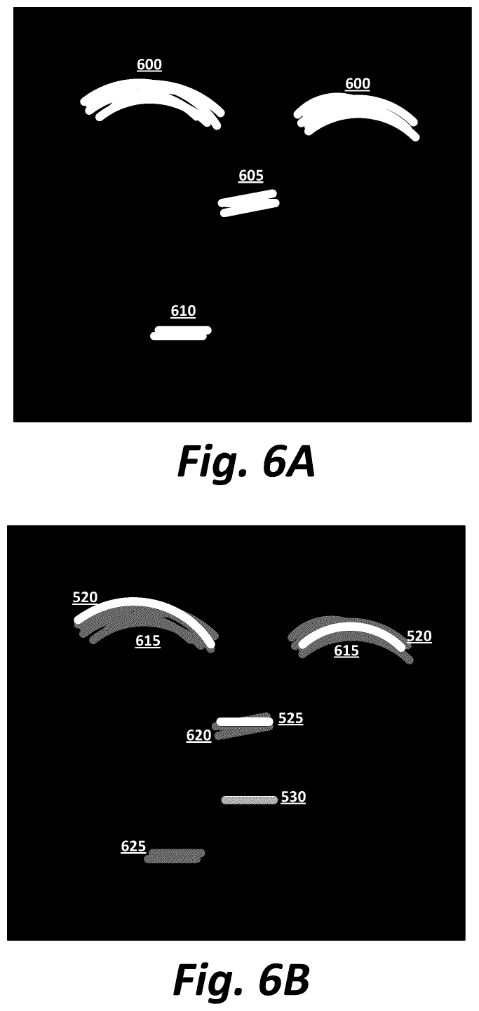

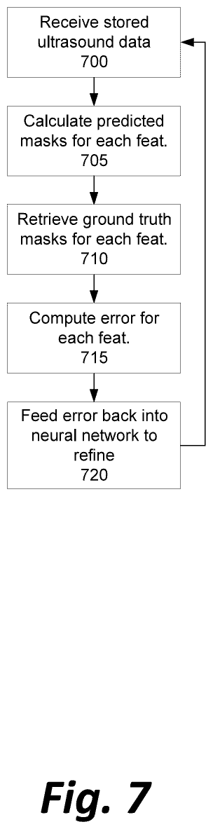

FIG. 5B is depicted with exclusively white markings for clarity in black-and-white reproduction. However, in a preferred embodiment, the human expert might annotate the images with multiple colors of stylus markings, each color being used to indicate different types of bone or anatomical features that the expert believes to be present. For example, the facet tracings 520 might be depicted in a first color, the posterior epidural tracings 525 might be depicted in a second color, and the anterior epidural tracings 530 might be depicted in a third color. The visual representation to the human annotator or a human reviewing the annotations at a later time are not as important as the fact that the annotation accurately tags these segments or arcs as a particular bone type or anatomical feature. These annotated images are later used as a “ground truth” data point for future training, according to a method depicted further in FIGS. 6A, 6B, and 7.

FIG. 6A depicts an example feature identification by a machine learning system, and FIG. 6B depicts an error determination step during training of the machine learning system.

Based on prior training, a neural network receives the raw image data of FIG. 5A and attempts to determine, for each pixel, whether the pixel represents part of a facet, part of a posterior or anterior epidural space, part of another anatomical feature that the neural network has been trained to detect, or not part of any relevant determination. As a result, one or more clouds of facet points 600, a possible one or more clouds of posterior epidural space points 605, and a possible one or more (supposed) clouds of anterior epidural space points 610 are output by the neural network.

After the machine learning output depicted in FIG. 6A has been generated, it is compared on a pixel-by-pixel basis with the human-annotated image depicted in FIG. 5B, which stores the denoted regions in the same format.

Where the original annotations and machine-generated annotations 520 and 600 or 525 and 605 overlap, a true positive region is recorded. Where a machine prediction of a region occurs that is imprecise (as with the clouds of close guesses 615 around each facet) or completely incorrect (as with the spurious identification 625 of an anterior epidural space), a false positive region is recorded. Where a machine prediction fails to identify a feature that a human identified, as with the missed anterior epidural space 530, a false negative region is recorded. Where the negative regions overlap (that is, the majority of each image, where both are in black), the true negative is recorded. These four sets of regions are used for back-propagating the error through the neural network underlying the machine classifier, to improve accuracy, as described further below.

FIG. 7 depicts, in simplified form, a method for evaluation of the machine learning output and back-propagating the error to reduce future error in classification.

Each iteration of training begins by receiving a raw ultrasound image that has been previously annotated by a human expert, as in FIG. 5B (Step 700). The raw ultrasound image is fed into the neural network, producing a mask output similar to that depicted in FIG. 6A (Step 705). Afterwards or in parallel, the human annotations for the same raw ultrasound that were previously recorded are retrieved from the database or other long term memory storing such annotations (Step 710). The error in the output is computed by comparing the two masks on a pixel-by-pixel basis, as depicted in FIG. 6B, to determine regions of true positive, false positive, false negative, and true negative (Step 715). Finally, the error is back propagated through the neural network to adjust the weightings of individual neurons and improve accuracy of future machine classifications. After the image has been used, the next previously-annotated ultrasound image is retrieved, and the process repeats (back to Step 700), until no more training data is available.

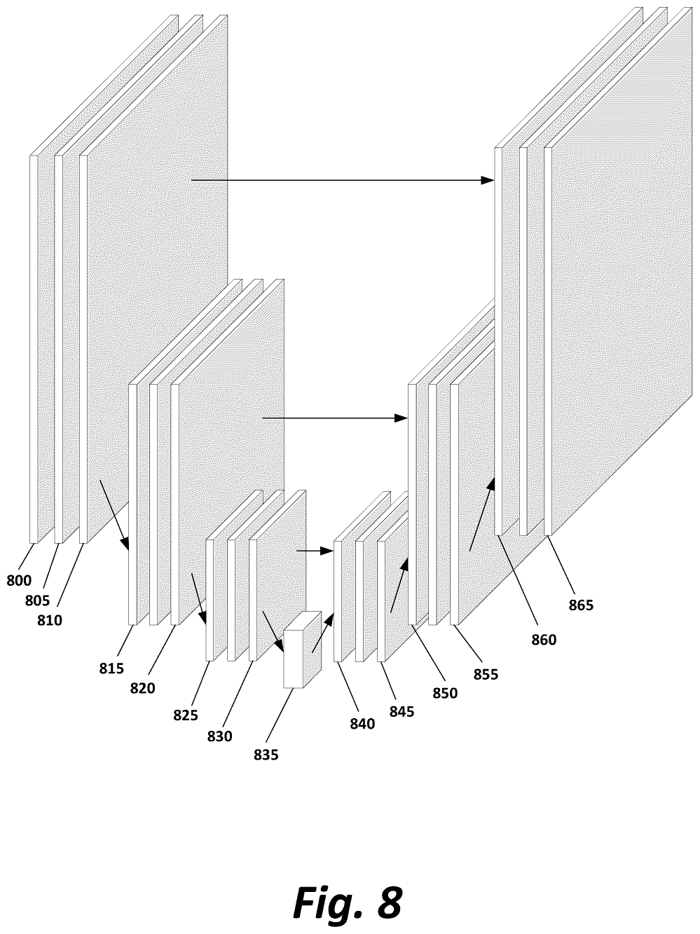

An example neural network structure that has been found to be particularly advantageous and effective is depicted in FIG. 8 and described below.

FIG. 8 depicts an exemplary neural network machine learning structure to be trained for use in structure identification and classification.

In a preferred embodiment, a neural network based on the U-Net architecture may be used. In other embodiments, alternatives that still incorporate convolutional neural networks may be used instead.

The input 800 to the neural network should be in the form of a 176 by 128 pixel image generated by ultrasound. In the preferred embodiment, it undergoes two convolution steps resulting in images 805 and 810 of identical or at least similar size—in many such convolutions, the edges are omitted at each convolution, reducing by two pixels in each dimension. After the convolutions, a downsampling step using the max pool function is performed, resulting in a much smaller image 815. Two more convolutions may be performed to obtain a further image 820. This process may be repeated at least once more, with downsampled step 825 and doubly-convolved step 830. In some embodiments, the steps are fully repeated a fourth time, but not depicted here.

Ultimately, after the repeated convolutions and max pool downsamplings are completed, a series of values 835 has been generated that is used to generate the feature-identifying masks via a series of upsampling and further convolutions.

A first upsampled mask 840 is generated based on input not only from the series of values 835 but also direct input from the last resulting image 830. That mask 840 is twice convolved to produce a mask 845. A second upsampled mask 850 is generated based on input not only from the mask 845 but also direct input from the second-to-last resulting image 820. That mask 850 is twice convolved to produce another mask 855. Finally, a third upsampled mask 860 is generated based on input not only from the mask 855 but also direct input from the third-to-last resulting image 810. If the neural network were to have a downsampling and upsampling “depth” of four or greater instead of three, this process would continue to generate the nth upsampled mask based on the n−1th convolved upsampled mask and the nth-to-last resulting image during the downsampling path.

Ultimately a mask 865 is generated based on all of the previous steps, having dimensions identical to the original input, and permitting the mask to be overlaid on an ultrasound image to show the features that have been identified by the neural network.

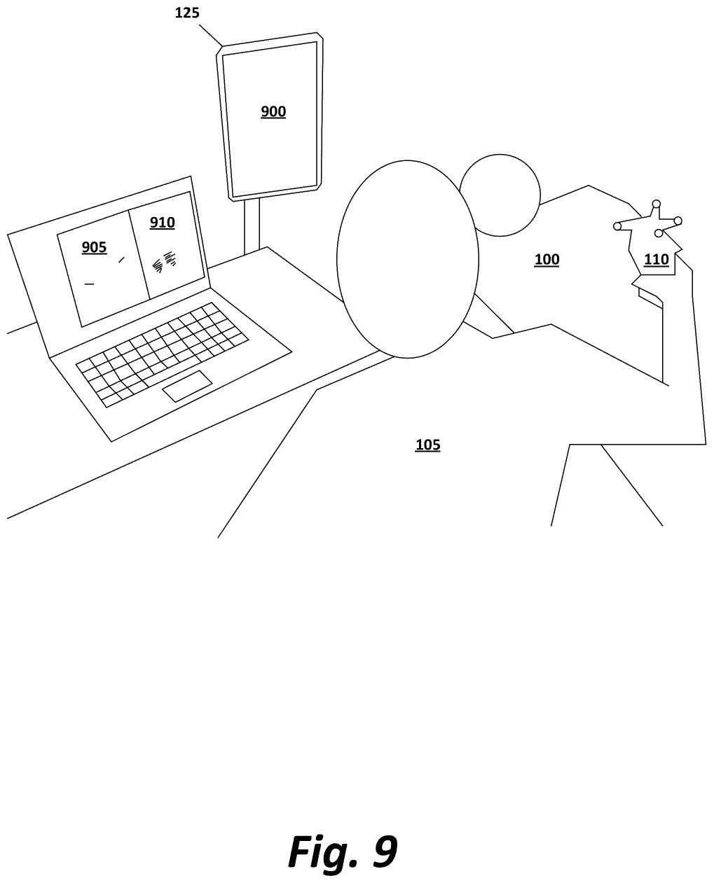

FIG. 9 depicts another example use of the system in a clinical environment for 3D ultrasound data generation.

As in FIG. 1, a human operator 105 uses an ultrasound probe 110 to scan the back of a patient 100 according to the three dimensional model embodiment depicted in FIGS. 2 and 3. At any given moment, three displays 900, 905, 910 are providing information to the operator. An ultrasound display 900 is shown on the display device 125 to display raw ultrasound data from the probe 110. On another display, such as a laptop computer, two windows may show additional information side-by-side. In the first display 905, the machine learning generated feature detection displays the currently identified features in real time from the ultrasound stream, as in FIG. 6A, or with those features overlaid on the ultrasound as a combination of FIG. 5A with FIG. 6A as a mask over it. In the second display 910, the detected features are stitched together to form a stable and increasingly detailed three-dimensional model as depicted in FIG. 3.

As previously mentioned, coloration is preferably used in the annotated displays 905 and 910 to inform the operator of the machine learning analysis's conclusions. For example, one preferred embodiment highlights spinous processes in magenta, lamina in cyan, facets in green, and transverse processes in yellow, to provide strong contrasts against a grayscale ultrasound underlying the display. In other embodiments, naturally, different color schemes might be used, and other information might be highlighted in addition to only those bony surfaces, such as indication of the acoustic window or other negative spaces. Additionally, different means other than coloration alone may be used to annotate the display with information derived from machine learning. If a display is monochrome, other visual texturing methods, such as cross-hatching, stippling, or other patterns may be used to indicate various surfaces or negative spaces. Alternatively, animation may be used, such as causing annotations for particular identified surfaces or negative spaces to flash, pulse, alternate colors, transition colors along a gradient, or otherwise catch the operator's eye.

As a result of these three generated displays, the operator 105 can view, side-by-side and in real time, (1) how the construction of the three-dimensional model is progressing, (2) whether an acoustic window is identified at the current location of the probe, and (3) what raw ultrasound data is currently being fed to the machine learning classifier.

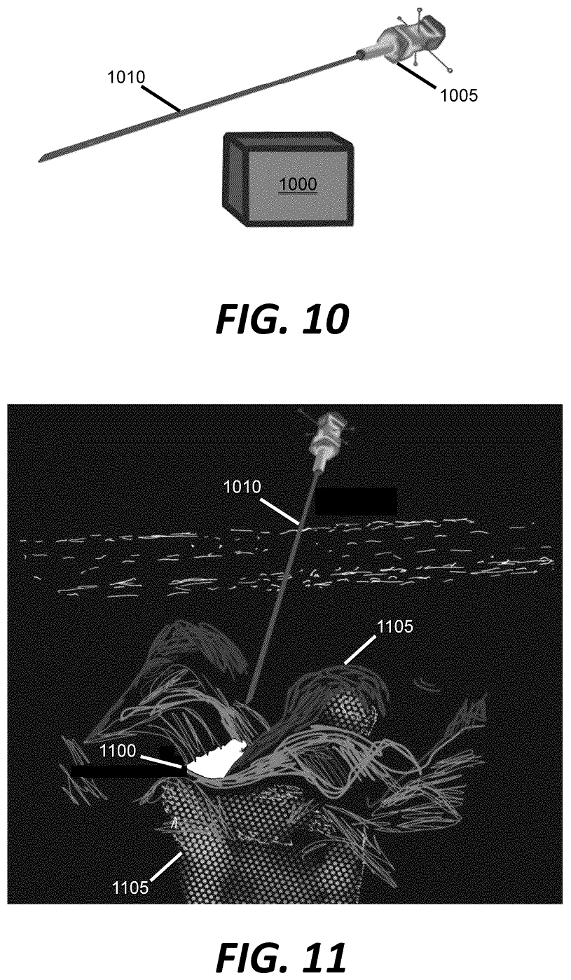

FIG. 10 depicts a navigated needle with EM tracker attached as the preferred way of live navigation of the needle with a user interface.

As depicted, an electromagnetic source 1000 generates electromagnetic radiation that is picked up by one or more electromagnetic sensors 1005. A needle 1010 is attached to the sensors and its location and orientation with respect to the source 1000 may be determined precisely.

FIG. 11 depicts the navigated needle displayed on the user interface in real time as it is seen traversing the soft tissues to enter the thecal sac through the acoustic window (seen on three-dimensional point cloud of bony surface) of a spine.

As depicted, the needle 1010 from FIG. 10 is injected into a patient into an acoustic window 1100, avoiding various other structures 1105 (shown here as a point cloud in one instance, and as a continuous approximation of a surface in another instance) that could cause serious injury to the patient or a failure of the injection if encountered by the needle 1010.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system for identifying acoustic windows in a spine of a patient, comprising:
an ultrasound probe with means for identifying position and orientation of the probe during use;
a server in communication with the ultrasound probe and receiving two-dimensional ultrasound images from the probe as well as a position and orientation of the probe when capturing each image; and
non-transitory memory storing instructions that, when executed by one or more processors of the server, cause the server to:
feed the two-dimensional ultrasound images into a trained neural network to identify bone and other features in the two-dimensional ultrasound images;
using the identified features and positions and orientations of the ultrasound probe at the moment the images were generated, generate a three-dimensional image of vertebrae in the spine;
identify an acoustic window between two vertebrae; and
guide a human user to penetrate with an injection needle at the acoustic window and to inject within a space in the spine while avoiding the injection needle encountering bone or neurovascular structure.

2. The system of claim 1, wherein the trained neural network is a convolutional neural network comprising at least two downsampling steps and at least two upsampling steps after the at least two downsampling steps.

3. The system of claim 1, wherein the trained neural network is trained via input of a series of ultrasound images that have been annotated by a human evaluator to indicate what regions of each ultrasound image correspond to which surfaces of a vertebra.

4. The system of claim 1, wherein the ultrasound probe comprises a needle guide, and wherein the guiding of the human user comprises a visual, auditory, or haptic feedback when the needle guide is aligned with the acoustic window such that passing the injection needle through the needle guide will cause the injection needle to avoid bone.

5. The system of claim 1, wherein the guiding of the human user comprises a visual or auditory message instructing the human user to move the ultrasound in a specified direction by a specified distance.

6. The system of claim 1, wherein the guiding of the human user comprises a visual or auditory message instructing the human user to rotate the ultrasound in a specified direction by a specified angle.

7. The system of claim 1, wherein a second trained neural network is trained to identify bone and other features in radio frequency (RF) ultrasound data or in-phase quadrature (IQ) ultrasound data instead of brightness mode (B mode) ultrasound data, and wherein the three-dimensional image of vertebrae in the spine is generated based on output from the second trained neural network.

8. The system of claim 1, wherein a navigated needle is introduced into a navigated three-dimension ultrasound field, wherein the navigated needle can be seen in a three-dimensional point cloud of a bony surface of the spine, and wherein the needle can be visualized on a user interface in real time as a tip of the needle enters a thecal sac.

9. The system of claim 1, wherein an injection being performed is selected from among a facet injection, vertebroplasty, nerve root block, or grey ramus block.

10. A system for identifying acoustic windows in a spine of a patient, comprising:
an ultrasound probe;
a server in communication with the ultrasound probe and receiving two-dimensional ultrasound images; and
non-transitory memory storing instructions that, when executed by one or more processors of the server, cause the server to:
feed the two-dimensional ultrasound images into a trained neural network to identify bone and other features in the two-dimensional ultrasound images;
using the identified features, identify a spinous process of a patient's vertebra;
receive medical and demographic data concerning the patient;
automatically determine a likely location of an acoustic window with respect to the spinous process; and
guide a human user to penetrate with an injection needle at the likely location of the acoustic window and to inject within a space in the spine that avoids the injection needle encountering bone or neurovascular structure.

11. The system of claim 10, wherein the trained neural network is a convolutional neural network comprising at least two downsampling steps and at least two upsampling steps after the at least two downsampling steps.

12. The system of claim 10, wherein the trained neural network is trained via input of a series of ultrasound images that have been annotated by a human evaluator to indicate what regions of each ultrasound image correspond to which surfaces of a vertebra.

13. The system of claim 10, wherein the ultrasound probe comprises a needle guide, and wherein the guiding of the human user comprises a visual, auditory, or haptic feedback when the needle guide is aligned with the acoustic window such that passing the injection needle through the needle guide will cause the injection needle to avoid bone.

14. The system of claim 10, wherein the guiding of the human user comprises a visual or auditory message instructing the human user to move the ultrasound in a specified direction by a specified distance.

15. The system of claim 10, wherein the guiding of the human user comprises a visual or auditory message instructing the human user to rotate the ultrasound in a specified direction by a specified angle.

16. The system of claim 10, wherein the medical data and demographic data comprises an identification of the sex of the patient.

17. The system of claim 10, wherein the medical data and demographic data comprises an identification of the age of the patient.

18. The system of claim 10, wherein the medical data and demographic data comprises an identification of the body mass index of the patient or an indication of a level of obesity of the patient.

19. The system of claim 10, wherein the medical data and demographic data comprises an identification of a region of the patient's spine being scanned.

20. The system of claim 10, wherein a second trained neural network is trained to identify bone and other features in radio frequency (RF) ultrasound data or in-phase quadrature (IQ) ultrasound data instead of brightness mode (B mode) ultrasound data, and wherein the three-dimensional image of vertebrae in the spine is generated based on output from the second trained neural network.
